(19) United States
(12) Patent Application Publication
(10) Pub. No.: US 2004/0002725 A1
(43) Pub. Date: Jan. 1, 2004

(54) Percutaneous dilational medical insertion device

(76) Inventors: Thomas J. Nicholson, York, PA (US); William Jay Nicholson, York, PA (US); W. Jay Nicholson, York, PA (US)

Correspondence Address: Salvatore Anastasi, Esquire, Barley, Snyder, Senft & Cohen, LLC, 126 East King Street, Lancaster, PA, 17602, US

(21) Appl. No.: 10/180,758

(22) Filed: Jun. 26, 2002

(51) Int. Cl. A61M 29/00

(52) U.S. Cl. 606/192; 128/207.29

(57) ABSTRACT

A percutaneous dilational medical insertion device and method comprising a needle removeably disposed in a catheter and a tubular member. The catheter has an inflatable balloon member disposed between a first catheter end and a second catheter end. The second catheter end having a flexible end portion. The needle has a tip that extends beyond the second catheter end that is percutaneously inserted into an interior cavity. After insertion into the interior cavity, the needle is removed from the catheter. When the needle is removed, the flexible end portion of the catheter curls to protect the interior cavity as the catheter is advanced. The catheter is advanced until the inflatable balloon member is received in a wall of the interior cavity. The inflatable balloon member is dilated to form a passageway. The tubular member disposed about the catheter proximate the first catheter end is then advanced into the passageway.

### FIELD OF THE INVENTION

The invention relates to a percutaneous dilational medical insertion device and, more particularly, to a percutaneous dilational medical insertion device having an inflatable balloon member for forming a passageway in a tracheal wall or skin tissue for insertion of a tubular member.

### BACKGROUND OF THE INVENTION

Percutaneous dilational medical insertion devices are commonly used to form a passageway in a tracheal wall or skin tissue for insertion of a tubular member. For example, percutaneous dilational medical insertion devices are commonly used to provide an air passageway when a patient's airway is obstructed by forming a passageway in the patient's tracheal wall for insertion of a tracheal tube. Other common usages for percutaneous dilational medical insertion devices include the formation of passageways for the placement of chest tubes, gastrectomy tubes, trocars for laprascopic surgery and intra-abdominal drains.

One common example of a percutaneous dilational medical insertion device and method used for forming a passageway in a tracheal wall is disclosed in U.S. Pat. No. 5,653,230. In U.S. Pat. No. 5,653,230, a slight incision is made with a scalpel through a patient's skin adjacent to the tracheal wall. A hollow needle is inserted through the incision and the tracheal wall until it is received in a tracheal passage. A wire guide is inserted into the hollow needle until it is received in the tracheal passage. The hollow needle is then withdrawn, leaving the wire guide inside the tracheal passage. A catheter attached to a dilator tube and provided with an inflatable balloon member is positioned over the wire guide. The catheter is advanced along the wire guide until the inflatable balloon member is received in the tracheal wall. A dilator is then activated providing a fluid supply through the dilator tube and into the inflatable balloon member. The inflatable balloon member expands to dilate a portion of the tracheal wall. The inflatable balloon member is then deflated by extracting the liquid to leave a passageway. A tracheal tube disposed on the dilator tube is then advanced along the catheter and received in the passageway. As the tracheal tube is received in the passageway, the deflated balloon member is advanced into the tracheal passage. The deflated balloon member and wire guide are then withdrawn from the tracheal tube, leaving the tracheal tube in the passageway.

The formation of a passageway by the conventional percutaneous dilational medical insertion device and method requires a significant number of intricate steps. Specifically, the known device and method requires an incision before insertion of the needle and the advancement of a wire guide before insertion of the catheter. The number and intricacy of the steps not only leaves room for error, but also the length of time needed to perform all of the steps is poorly suited to an emergency situation where prompt restoration of the patient's air passageway is critical. It is therefore desirable to develop a percutaneous dilational medical insertion device and method that can atraumatically form a passageway for a tubular member in an integral step to eliminate error and conserve time.

### SUMMARY OF THE INVENTION

The invention relates to a percutaneous dilational medical insertion device having a needle removeably disposed in a catheter. The catheter having an inflatable balloon member disposed between a first catheter end and a second catheter end. The needle having a tip that extends beyond the second catheter end. The second catheter end having a flexible end portion that curls into an essentially j-shape when the needle is removed from the catheter.

The invention further relates to a percutaneous method for forming a passageway for insertion of a tubular member. A tip end of a needle removeably disposed in a catheter provided with an inflatable balloon member is percutaneously inserted through a wall of an interior cavity and into the interior cavity. The needle is withdrawn from the catheter leaving a flexible end portion of the catheter disposed in the interior cavity. The flexible end portion of the catheter curls into an essentially j-shape when the needle is removed from the catheter to protect the interior of the interior cavity as the catheter is advanced. The catheter is advanced until the inflatable balloon member is received in the wall of the interior cavity. The inflatable balloon member is dilated to form a passageway in the wall of the interior cavity for insertion of a tubular member.

### BRIEF DESCRIPTION OF THE DRAWINGS

FIG. 1 is a side view of a percutaneous dilational medical insertion device.

FIG. 2 is a side view of a catheter of the device.

FIG. 3 is a side view of a needle of the device.

FIG. 4 shows the insertion of the device into a tracheal passage.

FIG. 5 shows a flexible end portion of the catheter curled in an essentially j-shape in the tracheal passage after the needle is withdrawn from the catheter.

FIG. 6 shows advancement of the flexible end portion of the catheter curled in an essentially j-shape in the tracheal passage.

FIG. 7 shows an inflatable balloon member received in a tracheal wall.

FIG. 8 shows advancement of a tubular member into a passageway in the tracheal wall formed by the inflatable balloon member.

FIG. 9 shows the tubular member positioned in the passageway before the catheter is withdrawn.

FIG. 10 shows the tubular member positioned in the passageway after the catheter is withdrawn.

### DETAILED DESCRIPTION OF THE INVENTION

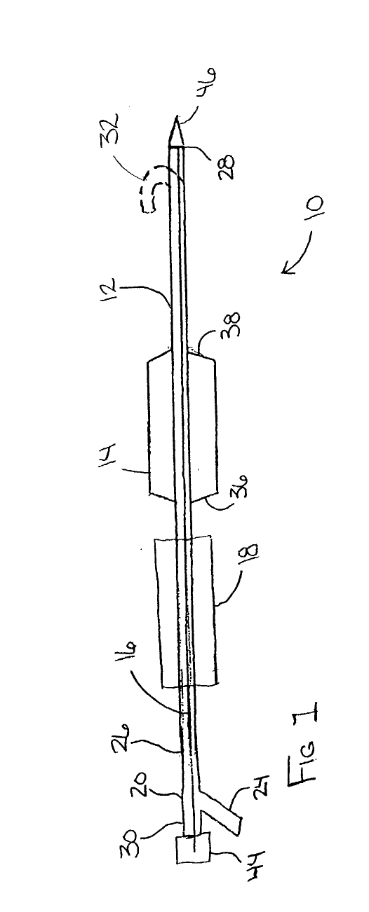

FIG. 1 shows a percutaneous dilational medical insertion device 10. The percutaneous dilational medical insertion device 10 comprises a catheter 12, a tubular member 18 and a needle 16. The needle 16 is removeably positioned within the catheter 12. The catheter 12 has an inflatable balloon member 14 and a first catheter end 26 having an attachment part 20 and a second catheter end 28 having a flexible end portion 32 that curls about the catheter 12 in an essentially j-shape (shown in phantom) when the needle 16 is removed. The tubular member 18 is positioned about the catheter 12.

The individual components of the device 10 will now be described in greater detail. Shown in FIG. 2, the catheter 12 comprises a flexible hollow tubular member extending from the first catheter end 26 to the second catheter end 28. The first catheter end 26 is provided with the attachment part 20. The attachment part 20 has a needle attachment portion 30 and a dilator attachment portion 24. The attachment part 20 may be integrally formed with the catheter 12 or may be a separate piece attached to the first catheter end 26. The second catheter end 28 has a flexible atraumatic end portion 32. The flexible end portion 32 essentially curls about the catheter 12 in an essentially j-shape. The length of the catheter 12 may vary depending upon the application, but a preferred length of the catheter 12 is essentially 17 cm.

Proximate the flexible end portion 32 is the inflatable balloon member 14. The inflatable balloon member 14 has an inflated periphery substantially similar to the size of the desired passageway. The inflatable balloon member 14 is essentially cylindrical in shape and has a first balloon end 36 and a second balloon end 38. The first balloon end 36 and the second balloon end 38 are tapered at an acute angle with respect to the longitudinal axis of the catheter 12. The inflatable balloon member 14 measures essentially 3 cm from the first balloon end 36 to the second balloon end 38. The first balloon end 36 is preferably positioned essentially 8 cm from the first catheter end 26 to ensure sufficient space between the dilator attachment portion 24 and the inflatable balloon member 14 for operation of a conventional dilator (not shown). The second balloon end 38 is preferably positioned essentially 6 cm from the second catheter end 28 to ensure proper advancement of the second catheter end 28 for positioning of the inflatable balloon member 14. It will be appreciated and understood by those in the art, however, that the dimensions and the positioning of the inflatable balloon member 14 may vary depending upon the desired size of the passageway and the medical application to obtain substantially similar results.

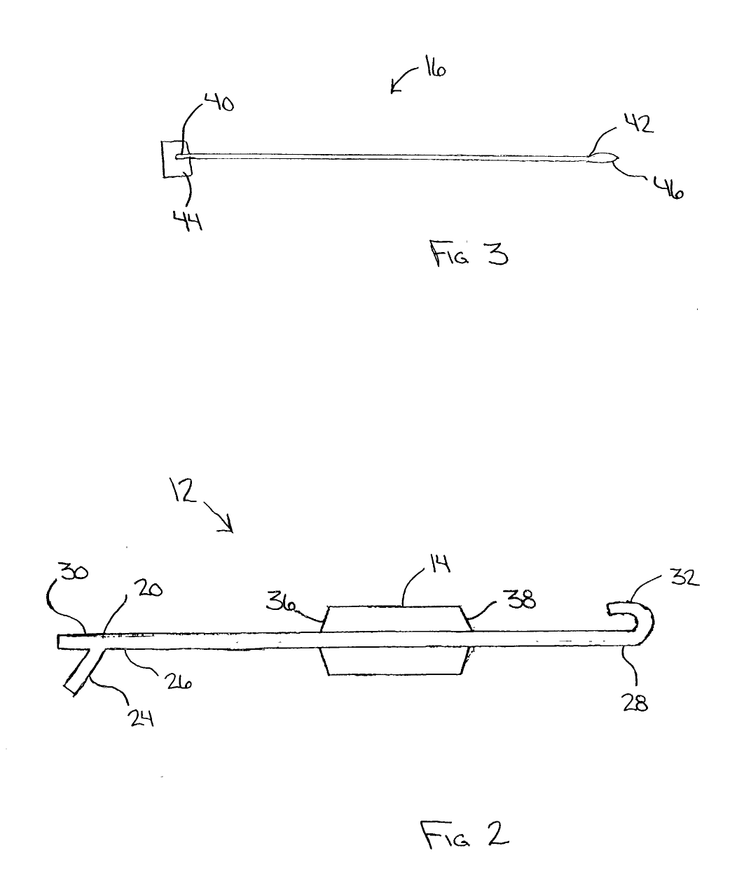

Shown in FIGS. 1 and 3, the needle 16 may be a conventional surgical needle having a diameter smaller than the internal diameter of the catheter 12. The needle 16 has a first needle end 40 and a second needle end 42. The first needle end 40 has an attachment receiving portion 44 for engagement with the needle attachment portion 30 of the attachment part 20 of the catheter 12. The second needle end 42 is formed to have an essentially pointed tip 46. The length of the needle 16 is such that the pointed tip 46 extends beyond the second catheter end 28 when the needle is removeably received in the catheter 12.

Shown in FIG. 1, the tubular member 18 may be a conventional medical tube suitable for use in medical applications, such as a tracheal tube or a chest tube. The tubular member 18 has an external diameter slightly smaller than the periphery of the inflated balloon member 14, and an internal diameter larger than the external diameter of the catheter tube 12. The tubular member 18 is positioned about the catheter 12 proximate the first catheter end 26 and adjacent to the first balloon end 36.

A method for forming a passageway for insertion of a tubular member using the device 10 will now be described with reference to FIGS. 4 through 10. Although the device 10 is illustrated as forming a passageway in a tracheal wall 50 for insertion of tubular member 18 in the form of a tracheal tube, it will be understood and appreciated by those skilled in the art that the device 10 may be used in a wide variety of medical applications and in conjunction with other body cavities.

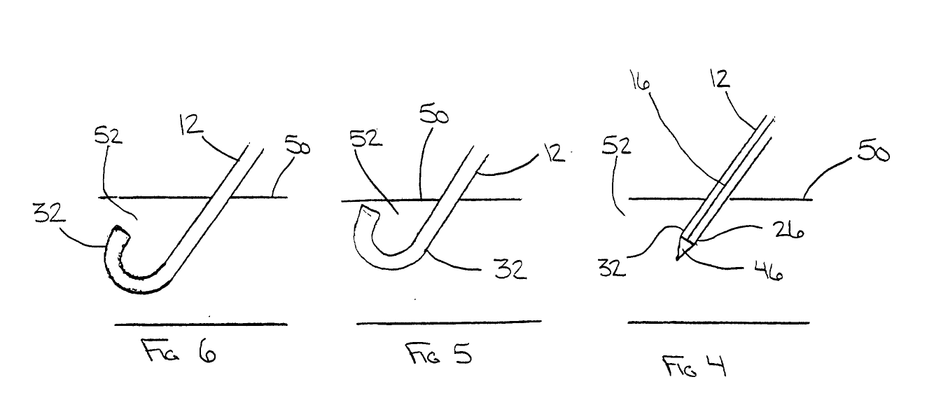

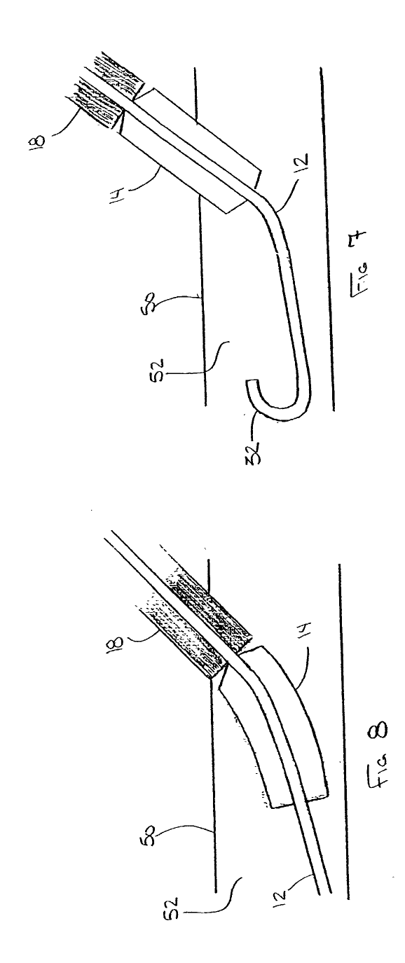

Shown in FIG. 4, the needle 16 is predisposed in the catheter 12 such that the pointed tip 46 of the needle 16 extends beyond the second catheter end 26 when the attachment receiving portion 44 is engaged with the needle attachment portion 30. The rigidity of the needle 16 causes the flexible end portion 32 to remain essentially straight for insertion of the pointed tip 46 of the needle 16 through the tracheal wall 50. As the needle 16 is inserted, the needle 16 punctures the skin adjacent to the tracheal wall 50 to create an opening. The pointed tip 46 of the needle 16 is inserted until the flexible end portion 32 of the second catheter end 28 is received in a tracheal passage 52. The needle 16 is then withdrawn from the catheter 12, leaving the flexible end portion 32 of the second catheter end 28 in the tracheal passage 52. As shown in FIG. 5, when the needle 16 is withdrawn, the flexible end portion 32 of the catheter 12 automatically curls to return to an essentially j-shape. Shown in FIGS. 6 and 7, the flexible end portion 32 is advanced inside the tracheal passage 52 until the inflatable balloon member 14 is received in the tracheal wall 50. Because the flexible end portion 32 has an essentially j-shape, advancement of the catheter 12 may be performed without damaging the interior of the tracheal wall 50.

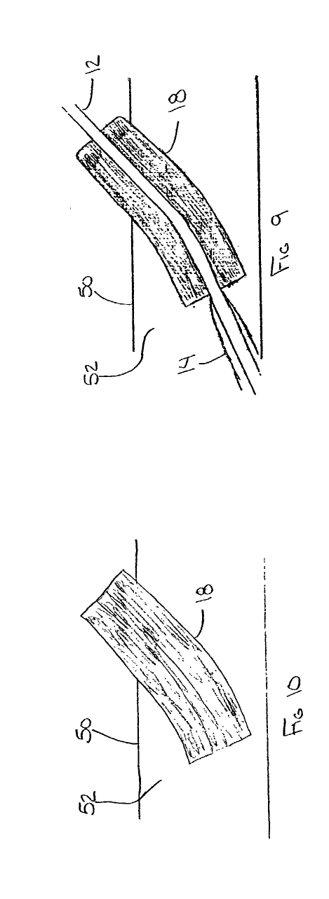

The conventional dilator (not shown) is activated to provide a fluid supply through a conventional dilator tube (not shown) engaged with the dilator attachment portion 24. The fluid supply is received in the second catheter end 28 and directed into the inflatable balloon member 14. Shown in FIG. 7, the inflatable balloon member 14 expands to dilate a portion of the tracheal wall 50 to form a passageway. After the inflatable balloon member 14 is inflated, the tubular member 18 is advanced along the catheter 12 toward the first balloon end 36, as shown in FIG. 8. The tubular member 18 contacts the first balloon end 36 and advances the first balloon end 36 into the tracheal passage 52 as the tubular member 18 is received in the passageway. As shown in FIGS. 9 and 10, the balloon member 14 is then deflated and the catheter 12 is withdrawn from the tubular member 18, leaving the tubular member 18 in the tracheal wall 50. Although the inflatable balloon member 14 is illustrated as being inflated when the tubular member 18 is advanced into the pasageway, it is well understood and appreciated by those skilled in the art that the inflatable balloon member 14 may also be deflated before advancement of the tubular member 18 into the passageway to obtain a substantially similar result.

While the device 10 is illustrated as being used in conjunction with the formation of a passageway in a tracheal wall for insertion of a tracheal tube, the device 10 may be used to form a passageway in any body tissue in a substantially similar manner. For example, the device 10 may be used in conjunction with other body cavities for insertion of other tubular members, such as chest tubes, gastrectomy tubes, trocars for laprascopic surgery and intra-abdominal drains.

The foregoing illustrates some of the possibilities for practicing the invention. Many other embodiments are possible within the scope and spirit of the invention. For example, although the attachment part is formed with a needle attachment portion and a dilator attachment portion, the attachment part may be formed with a single attachment portion that receives the needle and attaches the conventional dilator such that after the needle is withdrawn from the single attachment portion of the catheter, the conventional dilator tube of the conventional dilator is attached to the single attachment portion to supply fluid to the inflatable balloon member. It is, therefore, intended that the foregoing description be regarded as illustrative rather than limiting, and that the scope of the invention is given by the appended claims together with their full range of equivalents.

What is claimed is:

1. A percutaneous dilational medical insertion device comprising:
a catheter having an inflatable balloon member disposed between a first catheter end and a second catheter end;
the second catheter end having a flexible end portion; and
a needle removeably disposed in the catheter having a tip that extends beyond the second catheter end.

2. The device of claim 1, wherein the flexible end portion of the second catheter end curls into an essentially j-shape when the needle is removed from the catheter.

3. The device of claim 1, further comprising a tubular member disposed about the catheter between the first catheter end and the inflatable balloon member.

4. The device of claim 1, wherein the needle has an attachment receiving portion distal from the tip and the first catheter end has an attachment part for engaging the attachment receiving portion.

5. The device of claim 1, wherein the first catheter end has an attachment part having a needle attachment portion for receipt of the needle and a dilator attachment portion for engaging a dilator tube.

6. The device of claim 1, wherein the catheter has a length of essentially 17 cm.

7. The device of claim 1, wherein the inflatable balloon member is positioned essentially 6 cm from the second catheter end.

8. A percutaneous dilational medical insertion device comprising:
a catheter having an inflatable balloon member disposed between a first catheter end and a second catheter end;
a needle removeably disposed in the catheter having a tip that extends beyond the second catheter end;
the second catheter end having a flexible end portion that curls into an essentially j-shape when the needle is removed from the catheter; and
a tubular member disposed about the catheter between the first catheter end and the inflatable balloon member.

9. The device of claim 8, wherein the needle has an attachment receiving portion distal from the tip and the first catheter end has an attachment part having a needle attachment portion for engaging the attachment receiving portion of the needle and a dilator attachment portion for engaging a dilator tube.

10. The device of claim 9, wherein the catheter has a length of essentially 17 cm.

11. The device of claim 10, wherein the inflatable balloon member has a length of essentially 3 cm and a first balloon end positioned essentially 8 cm from the first catheter end and a second balloon end positioned essentially 6 cm from the second catheter end.

12. The device of claim 9, wherein the inflatable balloon member is positioned essentially 6 cm from the second catheter end.

13. The device of claim 8, wherein the first catheter end has an attachment portion for engaging a dilator tube.

14. A percutaneous method for forming a passageway for insertion of a tubular member, comprising the steps of:
percutaneously inserting a tip end of a needle removeably disposed in a catheter provided with a inflatable balloon member through a wall of an interior cavity and into the interior cavity;
withdrawing the needle from the catheter leaving the catheter disposed in the interior cavity;
advancing the catheter until the inflatable balloon member is received in the wall of the interior cavity; and
dilating the inflatable balloon member to form a passageway in the wall of the interior cavity.

15. The method of claim 14, further comprising the steps of:
advancing a tubular member into the wall of the interior cavity such that the inflatable balloon member is received in the interior cavity;
deflating the inflatable balloon member; and
withdrawing the inflatable balloon member and the catheter from the interior cavity leaving the tubular member in the wall of the interior cavity.

16. The method of claim 15, wherein the catheter has a flexible end portion that curls into an essentially j-shape when the needle is removed from the catheter to protect the interior of the interior cavity.

17. The method of claim 16, wherein the tubular member has an external diameter smaller than the diameter of the inflatable balloon member when the inflatable balloon member is dilated.

18. The method of claim 16, wherein the wall is a tracheal wall, the interior cavity is the interior of the tracheal wall and the tubular member is a tracheal tube.

19. The method of claim 18, wherein the catheter has a length of essentially 17 cm and the inflatable balloon member is positioned essentially 6 cm from the flexible end portion of the catheter.

20. The method of claim 18, wherein the catheter has a length of essentially 17 cm and the inflatable balloon member has a first balloon end positioned essentially 8 cm from a first positioned essentially 6 cm from the flexible end
